SEQUENCE LISTING (2 sequences)

SEQ ID NO: 1 - DNA, 20 bp, Artificial Sequence
  Description of Artificial Sequence Synthetic primer
accgcgctgagatctaaaaa

SEQ ID NO: 2 - DNA, 20 bp, Artificial Sequence
  Description of Artificial Sequence Synthetic primer
ctgcaaaggggtcagaagag

(19) United States
(12) Patent Application Publication
(10) Pub. No.: US 2016/0015701 A1
(43) Pub. Date: Jan. 21, 2016

(54) Methods of Treating Acute Kidney Injury

(71) Applicants: ABBIVE INC., North Chicago, IL (US); FRED HUTCHINSON CANCER RESEARCH CENTER, Seattle, WA (US)

(72) Inventors: Richard A. Zager, Mercer Island, WA (US); Dennis Andress, Chicago, IL (US)

(21) Appl. No.: 14/772,621

(22) PCT Filed: Mar. 10, 2014
(86) PCT No.: PCT/US2014/022688

§ 371 Date: Sep. 3, 2015

Related U.S. Application Data
(60) Provisional application No. 61/775,174, filed on Mar. 8, 2013.

(51) Int. Cl. A61K 31/454; G01N 33/68; C12Q 1/68; A61K 31/4025

(52) U.S. Cl. CPC A61K 31/454; A61K 31/4025; G01N 33/6893; C12Q 1/6883; G01N 2800/347; G01N 2800/7038; G01N 2800/7019

(57) ABSTRACT

Methods are provided for treating acute kidney injury in a subject, particularly ischemia-induced kidney injury and/or hypoxia-induced kidney injury. The methods comprise administering to the subject an ETA receptor antagonist, such as atrasentan or a pharmaceutically acceptable salt thereof. Methods of diagnosing and treating such kidney injuries are also provided. Methods of reducing or preventing loss of kidney function and/or renal mass or volume, and methods of delaying progression to chronic kidney disease are also provided.

### STATEMENT REGARDING FEDERALLY SPONSORED RESEARCH OR DEVELOPMENT

This invention was made with government support under grants (DK38432 and DK083310) awarded by the National Institutes of Health. The government has certain rights in the invention.

### TECHNICAL FIELD

The present disclosure is directed to methods for treating or preventing acute kidney injury.

### BACKGROUND OF THE INVENTION

Recent studies have reported an increase in hospitalizations due to acute kidney injury (AKI). (Waiker S S., et al., Declining mortality in patients with acute renal failure, 1988 to 2002. J Am Soc Nephrol 17: 1143-1150, 2006; Xue J L, et al., Incidence and mortality of acute renal failure in Medicare beneficiaries, 1992 to 2001. J Am Soc Nephrol 1135-1142, 2006.) The incidence of morbidity and mortality is high in these patients, and there is an urgent need for an effective therapy.

Acute kidney injury occurs when one or both kidneys are injured from one or more various causes and may result in a rapid loss of kidney function such as filtering waste products from blood. Causes of AKI include, but are not limited to, (1) exposure to a nephrotoxic agent; (2) systemic inflammatory response syndrome due to trauma, burns, pancreatitis, sepsis, or infection; (3) any physiologic condition that results in low blood volume, including peripheral arterial occlusive disease, arteriosclerosis obliterans, low cardiac output, volume redistribution, or altered vascular resistance; (4) traumatic rhabdomyolysis; (5) persistence or aggravation of inflammatory cytokinemia; (6) obstruction of the urinary tract; and (7) other intrinsic renal causes of acute kidney injury. Other diseases and conditions which place a subject at risk of AKI include: kidney transplantation surgery (as donor or recipient), bilateral arterial occlusion, bilateral acute renal vein thrombosis, acute uric acid nephropathy, hypovolemia, cardiovascular collapse, acute bilateral upper tract obstruction, hypercalcemic nephropathy, hemolytic uremic syndrome, acute urinary retention, malignant nephrosclerosis, essential mixed cyroimmunoglobulinemia, oxalate nephropathy, cortical necrosis, postpartum glomerulosclerosis, hypersensitivity nephropathy, scleroderma, idiopathic rapidly progressive glomerulonephritis, Goodpasture's syndrome, non-Goodpasture's anti-GBM disease, acute bacterial endocarditis or visceral sepsis, microscopic polyarteritis nodosa, Wegener's granulomatosis, allergic granulomatosis, acute radiation nephritis, post-streptococcal glomerulonephritis, nonstreptococcal post-infectious glomerulonephritis, diffuse proliferative lupus nephritis, membranoproliferative glomerulonephritis, renal vein thrombosis, Waldenstrom's macroglobulinemia, multiple myeloma, Berger's (IgA) nephropathy, Henoch-Schönlein purpura, and focal glomerulosclerosis. The result of AKI is that waste products begin to accumulate in the bloodstream, which can ultimately cause a number of complications including metabolic acidosis, hyperkalemia, uremia, hypovolemia, edema and death.

Because the etiology of AKI is diverse, a standard pharmacological treatment of AKI does not exist. When faced with a patient suffering from AKI, physicians attempt to manage the disease with fluid modulation and treat the underlying cause to minimize renal damage. A myriad of different types of compounds have been investigated to treat acute kidney injury with limited success. Proposed treatments of AKI include diuretics, caspase inhibitors, minocycline, guanosine, pifithrin-alpha, PARP inhibitors, sphingosine 1 phosphate analogs, adenosine 2A agonists, alpha-MSH, IL-10, fibrate, PPAR-gamma agonists, activated C protein, iNOS inhibitors, insulin, ethyl pyruvate, recombinant EPO, hepatocyte growth factor, carbon monoxide release compound and bilirubin, fenoldopam, and atrial natriuretic peptide.

Several therapies have shown preclinical promise, but have failed when investigated in a clinical setting, in part, because the therapeutic window for prevention of AKI is likely to be narrow, and delayed treatment is likely to be ineffective. (Jo S K, et al., Pharmacologic Treatment of Acute Kidney Injury: Why Drugs Haven't Worked and What is on the Horizon. Clin J Am Soc Nephrol 2: 356-365, 2007). For example, recombinant human IGF-1 (Miller S B, et al., Insulin-like growth factor I accelerates recovery from ischemic acute tubular necrosis in the rat. Proc Natl Acad Sci USA 89: 11876-11880, 1992) and atrial natriuretic peptide (Allgren R L, et al., Anaritide in acute tubular necrosis. Auriculin Anaritide Acute Renal Failure Study Group. N Engl J Med 336: 828-834, 1997; and Lewis J, et al., Atrial natriuretic factor in oliguric acture renal failure. Auriculin Anaritide Acute Renal Failure Study Group. Am J Kidney Dis 36: 767-774, 2000) have both failed in human trials.

AKI can be clinically diagnosed and evaluated by assessing certain laboratory parameters (e.g., serum creatinine (SCr), glomerular filtration rate (GFR), blood urea nitrogen (BUN), markers of inflammation). Kidney injury resulting from AKI can further be assessed via certain biomarkers, including kidney injury molecule 1 (KIM-1), human neutrophil gelatinase-associated lipocalin (NGAL), interleukin-18 (IL-18), cystatin C, clusterin, fatty acid binding protein, and osteopontin. (Cruz D N, et al., Neutrophil gelatinase-associated lipocalin as a biomarker of cardiovascular disease: a systematic review. Clin Chem Lab Med. 50: 1533-1545, 2012.)

A growing amount of clinical literature indicates that acute kidney injury can also initiate the onset of chronic kidney disease. (Ishani A, et al., Acute kidney injury increases risk of ESRD among elderly. J Am Soc Nephrol 20:223-228, 2009; Xue J L, et al., Incidence and mortality of acute renal failure in Medicare beneficiaries, 1992 to 2001. J Am Soc Nephrol 17: 1135-1142, 2006; Waikar S S, et al., Declining mortality in patients with acute renal failure, 1988 to 2002. J Am Soc Nephrol 17:1143-50, 2006; Liangos O, et al., Epidemiology and outcomes of acute renal failure in hospitalized patients: a national survey. Clin J Am Soc Nephrol 1: 43-51, 2006; Wald R, et al., Chronic dialysis and death among survivors of acute kidney injury requiring dialysis. JAMA 302: 1179-85, 2009; Goldberg A, et al., The impact of transient and persistent acute kidney injury on long-term outcomes after acute myocardial infarction. Kidney Int 76: 900-910, 2009.) For example, a recent study reported that patients who required dialysis due to AKI had a ˜28 fold increased risk of developing chronic kidney disease (CKD). (Lo L J, et al., Dialysis-requiring acute renal failure increases the risk of progressive chronic kidney disease. Kidney Int. 76: 893-9, 2009.) However, the mechanisms by which AKI might initiate the onset of CKD have not been identified with certainty.

One prominent theory holds that an initial ischemic insult can induce peritubular microvascular damage, thereby compromising renal blood flow. This may culminate in persistent renal ischemia with ongoing tissue damage due to hypoxic conditions. (Basile D P, et al., Impaired endothelial proliferation and mesenchymal transition contribute to vascular rarefaction following acute kidney injury. Am J Physiol 300: F721-733, 2011; Leonard E C, et al., VEGF-121 preserves renal microvessel structure and ameliorates secondary renal disease following acute kidney injury. Am J Physiol 295: F1648-1657, 2008.) Ischemia occurs when there is insufficient blood flow to provide adequate oxygenation, which results in tissue hypoxia (reduced oxygen) or anoxia (absence of oxygen) as the most severe form of hypoxia, and ultimately tissue necrosis, and to a lesser extent apoptosis. Ischemia always results in hypoxia; however, hypoxia can occur without ischemia if, for example, the oxygen content of the arterial blood decreases, such as occurs with anemia. Therefore, a therapy that is efficacious in models of ischemia-induced kidney injury would also be efficacious in models of hypoxia-induced kidney injury because both models cause tissue damage by depriving the tissue of essential nutrients and by causing endothelial dysfunction, oxidative stress, and inflammation.

One of the factors that has contributed to the difficulty in defining the mechanism of acute kidney injury resulting from ischemia is the fact that the most commonly used model of ischemic AKI, bilateral renal artery occlusion (RAO), does not reliably produce progressive renal damage. Rather, despite the fact that RAO produces so called “healing defects” (e.g., persistent tubular/microvascular damage; salt sensitive hypertension), neither sustained nor progressive losses of GFR result. (See, e.g., Pechman K R, et al., Recovery from renal ischemia-reperfusion injury is associated with altered renal hemodynamics, blunted pressure natriuresis, and sodium-sensitive hypertension. Am J Physiol 297:R1358-R1363, 2009; Spurgeon-Pechman K R, et al., Recovery from acute renal failure predisposes hypertension and secondary renal disease in response to elevated sodium. Am J Physiol 293: F269-F278, 2007; Finn W F, et al., Recovery from postischemic acute renal failure in the rat. Kidney Int 16: 113-123, 1979; Finn W F, et al., Attenuation of injury due to unilateral renal ischemia: delayed effects of contralateral nephrectomy. J Lab Clin Med 103: 193-203, 1984.)

Recently a new model of unilateral ischemic injury (rather than bilateral) in the mouse was reported to produce ongoing tubular necrosis, interstitial inflammation, peritubular microvascular injury, renal fibrosis, and ultimately a 40-50% loss of renal mass over 2-3 weeks. (Zager R A, et al., Acute unilateral ischemic renal injury induces progressive renal inflammation, lipid accumulation, histone modification, and “end-stage” kidney disease. Am J Physiol 301: F1334-1345, 2011.) The presence of an uninjured contralateral kidney in the model improves mortality rates due to uremia and can allow investigators to more fully investigate acute kidney injury. The model of unilateral ischemic kidney injury results in a near ‘end stage’ renal disease in a matter of weeks. By using this unilateral ischemia model, certain several important pathophysiologic events that participate in progressive renal damage have been reported. These include stepwise increases in serum creatinine, BUN, pro-inflammatory cytokine generation, down regulation of selected anti-inflammatory defenses (e.g. heme oxygenase 1 and IL-10), and cumulative lipotoxicity. These culminate in progressive tubule necrosis, tubule dropout, early fibrosis, and ultimately a profound loss of renal mass. (Zager R A, et al., Acute unilateral ischemic renal injury induces progressive renal inflammation, lipid accumulation, histone modification, and “end-stage” kidney disease. Am J Physiol 301: F1334-1345, 2011.)

There is therefore a need for effective and safe therapy for treating acute kidney injury.

### SUMMARY OF THE INVENTION

The present disclosure is directed to methods of treating acute kidney injury with an endothelin receptor antagonist, such as atrasentan. As one aspect of the present invention, methods of treating acute kidney injury in a subject are provided. The methods comprise administering to the subject an ETA receptor antagonist, such as atrasentan or a pharmaceutically acceptable salt thereof. In particular, the methods are suitable for treating kidney injury that is an ischemia-induced kidney injury or hypoxia-induced kidney injury. The ETA receptor antagonist may be administered after the kidney injury, for example at least 24 hours after the kidney injury and/or after the subject develops clinical acute renal failure. In some embodiments, the ETA receptor antagonist is not administered to the subject before the onset and/or diagnosis of acute kidney injury, and in some embodiments, the methods exclude such administration.

As another aspect of the present invention, methods of treating ischemia-induced renal injury or hypoxia-induced renal injury in a subject are provided. As yet another aspect of the present invention, methods of delaying progression to chronic kidney disease in a subject having ischemia-induced kidney injury or hypoxia-induced kidney injury are provided. As still another aspect of the present invention, methods of reversing post-ischemic or post-hypoxic kidney damage in a subject are provided. In each of these aspects, the methods comprise administering to the subject an ETA receptor antagonist.

As yet another aspect of the present invention, methods of reducing the loss of renal mass in a subject having an ischemia-induced kidney injury or hypoxia-induced kidney injury are provided. The methods comprise diagnosing the subject as having acute kidney injury; making a first measurement of an AKI indicator; administering to the subject an ETA receptor antagonist; and making a later second measurement of the AKI indicator after the subject has been administered the ETA receptor antagonist for a period of time. The difference between the first measurement and second measurement of the AKI indicator is not significant. In some embodiments, the AKI indicator is kidney mass, kidney volume, glomerular filtration rate, serum creatinine, blood urea nitrogen, or markers of inflammation.

As another aspect of the present invention, methods of diagnosing and treating acute kidney injury in a subject are provided. The methods comprise measuring a level of an indicator of ischemia-induced renal injury or hypoxia-induced renal injury; determining whether the measured level indicates ischemia-induced renal injury or hypoxia-induced renal injury; and administering to the subject suffering from ischemia-induced renal injury or hypoxia-induced renal injury an ETA receptor antagonist. For example, indicators of ischemia-induced renal injury or hypoxia-induced renal injury include urinary tubular injury residue (i.e., urine samples showing tubular injury residue (e.g., tubular cell casts)), ET-1 mRNA levels expressed in the kidney, ETA receptor mRNA levels expressed in the kidney, NGAL mRNA levels expressed in the kidney, lactate, or markers of inflammation. Specifically, the mRNA expression may be localized within the renal cortex of the kidney. Other indicators may be protein levels of ET-1 or NGAL or other measures of ETA receptor expression or presence on cell surfaces.

In the foregoing methods, the ETA receptor antagonist may be atrasentan or a pharmaceutically acceptable salt thereof. The ETA receptor antagonist can be administered after the kidney injury, for example at least 24 hours after the kidney injury, or after the subject develops clinical acute renal failure.

### BRIEF DESCRIPTION OF THE FIGURES

FIG. 1 depicts renal cortical and plasma endothelin 1 mRNA levels following ischemic kidney injury.

FIG. 2 depicts renal cortical and plasma endothelin 1 protein levels at two weeks post-ischemic kidney injury.

FIG. 3 illustrates ChIP assay assessments of Pol II binding, histone 3 methylation, acetylation, and histone variant H2.Z exchange at exon 1 of the ET-1 gene.

FIG. 4 depicts renal cortical ETA and ETB receptor mRNA levels at 24 hrs and two weeks post ischemic injury.

FIG. 5 shows renal weights two weeks after the induction of unilateral ischemic injury+/−atrasentan treatment in the pre+post (left panel), or just the post ischemic period (right panel).

FIG. 6 shows photographs of the kidneys whose weights are shown in FIG. 5.

FIG. 7 depicts renal proliferation following the unilateral ischemic protocol with and without atrasentan treatment. The left hand panel depicts the weights of the contralateral (non ischemic) kidneys from the unilateral ischemic injury experiments ±atrasentan treatment. The right hand panel depicts KI-67 staining of a normal kidney (A), a 2 week (left) post ischemic kidney (B), and a 2 week left post ischemic kidney with pre+post atrasentan treatment (C).

FIG. 8 illustrates the effect of atrasentan on blood urea nitrogen (BUN) and plasma creatinine twenty-four hours after ischemia of different durations.

FIG. 9 illustrates the effect of atrasentan on NGAL twenty-four hours after ischemia of different durations.

### DETAILED DESCRIPTION

Section headings as used in this section and the entire disclosure herein are not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used herein, the term “about” is used synonymously with the term “approximately.” Illustratively, the use of the term “about” indicates that values slightly outside the cited values, namely, plus or minus 10%. Such dosages are thus encompassed by the scope of the claims reciting the terms “about” and “approximately.”

The terms “administer”, “administering”, “administered” or “administration” refer to any manner of providing a drug (such as astrasentan or a pharmaceutically acceptable salt thereof) to a subject or patient. Routes of administration can be accomplished through any means known by those skilled in the art. Such means include, but are not limited to, oral, buccal, intravenous, subcutaneous, intramuscular, transdermal, by inhalation and the like.

The term “active agent” as used herein refers to an agent that achieves a desired biological effect or a pharmaceutically acceptable salt thereof. The term “active agent” and “drug” are used interchangeably herein. The solid state form of the active agent used in preparing the dosage forms of the present disclosure is not critical. For example, active agent used in preparing the dosage forms of the present disclosure can be amorphous or crystalline. The final dosage form contains at least a detectable amount of crystalline active agent. The crystalline nature of the active agent can be detected using powder X-ray diffraction analysis, by differential scanning calorimetry or any other techniques known in the art.

The term “atrasentan” or “atra” OR “ABT-627” refers to (2R,3R,4S)-4-(1,3-benzodioxol-5-yl)-1-[2-(dibutylamino)-2-oxoethyl]-2-(4-methoxyphenyl)pyrrolidine-3-carboxylic acid having the structure shown below:

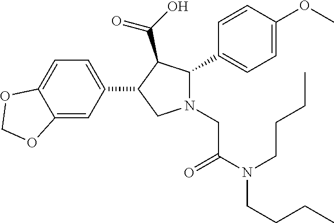

and salts thereof such as the HCl salt of atrasentan. Methods for making atrasentan are described, for example, in U.S. Pat. Nos. 6,380,241, 6,946,481, 7,365,093, 5,731,434, 5,622,971, 6,462,194, 5,767,144, 6,162,927 and 7,208,517, the contents of which are herein incorporated by reference.

The term BQ-788 refers to N-cis-2,6-dimethylpiperidinocarbonyl-L-gamma-methylleucyl-D-1-methoxycarbonyltryptophanyl-D-norleucine having the structure shown below:

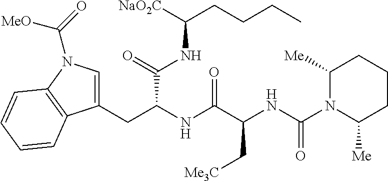

By an “effective amount” or a “therapeutically effective amount” of an active agent is meant a nontoxic but sufficient amount of the active agent to provide the desired effect. The amount of active agent that is “effective” will vary from subject to subject, depending on the age and general condition of the individual, the particular active agent or agents, and the like. Thus, it is not always possible to specify an exact “effective amount.” However, an appropriate “effective amount” in any individual case may be determined by using routine experimentation.

Of course, it will be understood that other dosage regimens may be utilized, such as dosing more than once per day, utilizing extended, controlled, or modified release dosage forms, and the like in order to achieve the desired result. It is contemplated that the ETA receptor antagonist may be administered upon detection of renal ischemia or hypoxia or within 24 hours after such detection. Alternatively or additionally, the ETA receptor antagonist may be administered for up to one week, or two weeks, or four weeks, or eight weeks, or longer. Or the ETA receptor antagonist may be administered for as long as the acute kidney injury exists or until the injury is resolved or until ischemia or hypoxia is no longer detected.

The term “endothelin subtype A receptor antagonist” or “ETA receptor antagonist” or “ETA receptor inhibitor” refers to any compound that inhibits the effect of ET-1 signaling through the endothelin subtype A receptor. Examples of ETA receptor antagonists include, but are not limited to, ambrisentan, atrasentan, avosentan, BMS 193884, BQ-123, CI-1020, clazosentan, darusentan, edonentan, S-0139, SB-209670, sitaxsentan, TA-0201, tarasentan, TBC 3711, tezosentan, YM-598, ZD-1611, ZD-4054, and salts, esters, prodrugs, metabolites, tautomers, racemates and enantiomers thereof. The term “endothelin antagonist” or “ET-1 antagonist” or “ET-1 inhibitor” refers to any compound that inhibits ET-1.

The term “acute kidney injury” or “acute kidney failure” is typically identified by a rapid deterioration in renal function sufficient to result in the accumulation of nitrogenous wastes in the body (see, e.g., Anderson and Schrier (1994), in Harrison's Principles of Internal Medicine, 13th edition, Isselbacher et al., eds., McGraw Hill Text, New York). Rates of increase in BUN of at least 4 to 8 mmol/L/day (10 to 20 mg/dL/day), and rates of increase of serum creatinine of at least 40 to 80 μmol/L/day (0.5 to 1.0 mg/dL/day), are typical in acute renal failure. Urinary samples also may contain tubular injury residue in patients suffering from acute kidney injury. In subjects which are catabolic (or hypercatabolic), rates of increase in BUN may exceed 100/mg/dL/day. Rates of increase in BUN or serum creatinine may be determined by serial blood tests and, preferably, at least two blood tests are conducted over a period of between 6 and 72 hours or, more preferably, 12 and 24 hours. A distinction is sometimes made between “acute” renal failure (deterioration over a period of days) and “rapidly progressive” renal failure (deterioration over a period of weeks). As used herein, however, the phrase “acute kidney injury” is intended to embrace both syndromes. Acute kidney injury is regularly identified by clinicians, as discussed above.

The term “ischemia-induced kidney injury” as used herein refers to renal injury due to one or more identified occurrences of renal ischemia or ischemia and reperfusion. The term ischemic kidney injury may be used interchangeably herein. The term “hypoxia-induced kidney injury” as used herein refers to renal injury due to ischemia, ischemia and reperfusion, and/or hypoxia, even if the hypoxia or apoxia is due to causes unknown or other than ischemia. Ischemia-induced kidney injury can be identified by clinicians, for example by recognizing ischemic conditions or by reference to certain intrinsic renal causes of acute kidney injury. Hypoxia-induced kidney injury can be identified by clinicians, for example by recognizing hypoxic conditions or by reference to certain intrinsic renal causes of acute kidney injury. The extent of the renal injury would be clinically assessed by tracking increases in BUN and serum creatinine.

The term “markers of inflammation” as used herein refers to peptides and chemicals produced by a patient's body when some aspect of the patient's body is in an inflammatory state. Exemplary markers of inflammation include triglycerides, non-high-density lipoprotein (HDL), apoprotein B, fibrinogen, soluble tumor necrosis factor receptor (sTNFR-2), monocyte chemoattractant protein-1, interferon-gamma-inducible protein (IP-10), macrophage inflammatory protein-1delta, vascular cell adhesion molecule-1 (VCAM), serum amyloid A, heme oxygenase 1, soluble intercellular molecule type 1 (sICAM-1), C-reactive protein (CRP) and members of the interleukin family. The presence of elevated levels of certain of these markers has been shown to be associated with development of disease. For example, CRP has been reported as a marker for systemic inflammation (Ridkler et al., C-Reactive Protein and Other Markers of Inflammation in the Prediction of Cardiovascular Disease in Women. 342(12):836-43, NEJM, 2000.). Markers of inflammation can be evaluated through collection of blood and/or urine.

The term “AKI indicator” as used herein refers to a clinical measurement that reflects the presence of acute kidney injury. Contemplated AKI indicators include certain laboratory parameters, including but not limited to, GFR, serum cystatin C, urinary albumin excretion, BUN, serum creatinine, urinary NGAL, urinary KIM-1, markers of inflammation and certain morphological markers, including, but not limited to, kidney mass and urine samples showing renal tubular cells and cellular residue.

The term “glomerular filtration rate” or “GFR” as used herein refers to actual or real glomerular filtration rate as well as estimations of glomerular filtration rate. Estimations of glomerular filtration rate can be made using certain equations including, the Modification of Diet in Renal Disease (MDRD) study equation, the Bedside Schwartz equation, the Cockcroft-Gault formula, or any other clinically acceptable formula or equation.

The term “clinical acute renal failure” as used herein refers to a decrease in renal function that is or can be detected clinically, such as by a general practitioner or a nephrologist. Clinical acute renal failure may be assessed by decreased glomerular filtration rate, decrease in or absence of urine production, increased waste products (especially creatinine or urea) in the blood, hematuria (blood loss in the urine), proteinuria (protein loss in the urine), or other clinical indicators.

The term “intrinsic renal causes of acute kidney injury,” as used herein, include:
(1) Abnormalities of the vasculature such as vasoconstrictive disease (e.g., malignant hypertension, scleroderma, hemolytic uremic syndrome, thrombotic thrombocytopenic purpura) and vasculitis (e.g., polyarteritis nodosa, hypersensitivity angiitis, serum sickness, Wegener's granulomatosis, giant cell arteritis, mixed cryoglobulinemia, Henoch-Schonlein purpura, systemic lupus erythematosus);
(2) Abnormalities of the glomeruli such as post-infectious abnormalities (e.g., post-streptococcal, pneumococcal, gonococcal, staphylococcal, enterococcal, viral [e.g., hepatitis B and C, mumps, measles, Epstein-Barr], malarial, or related to brucellosis, Legionella, Listeria, shunt nephritis, leprosy, leptospirosis, or visceral abscesses) and non-infectious abnormalities (e.g., rapidly progressive glomerulonephritis, membranoproliferative glomerulonephritis, Goodpasture's syndrome, systemic lupus erythematosus, Wegener's granulomatosis);
(3) Acute interstitial nephritis resulting from drug related causes (e.g., penicillins, sulfonamides, carbenicillin, cephalosporin, erythromycin, nafcillin, oxacillin, nonsteroidal antiinflammatory agents, diuretics (furosemide, ethacrynic acid, thiazide, spironolactone, mercurials), phenytoin, phenobarbital, probenicid, allopurinol, cimetidine), infection related causes (e.g., acute pyelonephritis, streptococcal, staphylococcal, leptospirosis, malaria, salmonellosis), papillary necrosis (e.g., associated with diabetes mellitus, sickle cell diseases, analgesic abuse, alcoholism), and other, miscellaneous causes (e.g., sarcoidosis, leukemia, lymphoma);
(4) Intratubular obstruction from crystal deposition (e.g., uric acid, oxalate, methotrexate) or multiple myeloma and light chain disease; and
(5) Acute tubular necrosis resulting from nephrotoxins (e.g., antimicrobials such as aminoglycosides, tetracyclines, amphotericin, polymyxin, cephalosporins), heavy metals (e.g., mercury, lead, arsenic, gold salts, barium), and other, miscellaneous chemical agents (e.g., cisplatin, doxorubicin, streptozocin, methoxyflurane, halothane, ethylene glycol, carbon tetrachloride), or from ischemia (e.g., hemorrhage, hypotension, sepsis, burns, renal infarction, renal artery dissection, rhabdomyolysis, trauma), or other miscellaneous causes (e.g., contrast agents, transfusion reactions, myoglobinemia, heat stroke, snake and spider bites).

By “pharmaceutically acceptable,” such as in the recitation of a “pharmaceutically acceptable excipient,” or a “pharmaceutically acceptable additive,” is meant a material that is not biologically or otherwise undesirable, i.e., the material may be incorporated into a pharmaceutical composition administered to a patient without causing any undesirable biological effects.

The term “RAAS inhibitor” refers to any compound that inhibits one or more elements of the renin-angiotensin-aldosterone system (RAAS). Examples of RAAS inhibitors include ACE inhibitors, ARBs, rennin inhibitors, aldosterone antagonists and others.

The term “subject” refers to an animal. In one aspect, the animal is a mammal, including a human or non-human, preferably a human subject. The terms patient and subject may be used interchangeably herein.

Preferably, a diagnosis that a subject is in acute renal failure, or at risk of entering acute renal failure, is made on the basis of serial blood tests measuring, among other factors, the circulating levels of serum creatinine and blood urea nitrogen. Such “serial” blood tests may be taken every few hours immediately upon admittance of an undiagnosed patient presenting with symptoms of acute renal failure. More typically, however, consecutive serial blood tests are separated by a period of at least 6 hours, not more than 72 hours, and preferably 12-24 hours. On the basis of two or more blood tests within a 24 or 72 hour period, it is possible to calculate a rate of increase of serum creatinine or BUN. Additionally, or alternatively, diagnosis can be made by assessing the presence of tubule injury residue in urinary samples.

Subjects possessing a single kidney, irrespective of the manner of loss of the other kidney (e.g., physical trauma, surgical removal, birth defect), may be considered to be at increased risk of acute renal failure. This is particularly true for those subjects in which one kidney has been lost due to a disease or condition which may afflict the remaining kidney. Similarly, subjects which are already recipients of a renal transplant, or which are receiving chronic dialysis (e.g., chronic hemodialysis or continuous ambulatory peritoneal dialysis) may be considered to be at increased risk of acute renal failure. Other groups that are at an increased risk of developing acute kidney injury include those with chronic kidney disease, diabetes mellitus, and the elderly. Therefore, for these subjects, the clinical indications discussed above may need to be more carefully monitored, and earlier or more aggressive intervention with renal therapeutic agent treatment may be advisable.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

The present methods are based, in part, upon the surprising discovery that therapeutic use of ETA receptor antagonists to subjects suffering from acute kidney injury, including after the onset or diagnosis of acute kidney injury, can reduce mortality and/or morbidity rates, and prevent, inhibit, delay, or alleviate the permanent and/or progressive loss of renal function associated with acute kidney injury. It is contemplated that the kidney injury can be hypoxia-induced kidney injury or, more specifically, an ischemia-induced kidney injury.

ETA receptor antagonism prevents or reduces physical kidney loss as well as functional renal loss associated with ischemic-induced and/or hypoxia-induced kidney injury. In a clinical setting, kidney loss can be evaluated by assessing kidney mass or kidney volume using ultrasound technology or other appropriate visualization technology.

In embodiments, renoprotective effects from the disclosed methods may occur before, during and/or after the initial ischemic/reperfusion injury phase. In embodiments, the ETA receptor antagonist is administered to accelerate renal recovery after AKI.

In embodiments, renoprotective effects from the disclosed methods result in a reduction in markers of inflammation and certain laboratory parameters that indicate kidney stress and dysfuntion (e.g., serum creatinine, GFR, BUN). Therefore, in some embodiments of the present invention, measurements of these markers of inflammation and/or certain laboratory parameters at some time after the administration of an ETA receptor antagonist show a reduction over time, which would correlate to an increase in renal function.

The exact mechanism of renoprotection afforded by ETA receptor antagonism is not clear. However, without being bound to any particular theory or mechanism, it is believed that the renoprotection may be driven by one or more of the following: direct ETA antagonism, interactions with nitric oxide signaling, the angiotensin II system, and TGFbeta-mediated fibrosis. If persistent tissue ischemia is, in fact, a mediator of progressive renal damage, as suggested above, then ET-1-mediated renal vasoconstriction could potentially play an important pathogenic role. It does appear that some of the renoprotective efficacy observed in ETA receptor antagonism is due to renal microvascular dilation and preservation of renal tubular cell structure and function. Therefore, in some embodiments of the present methods, a combination of an ETA receptor antagonist and RAAS inhibitor may be administered to provide added renoprotection over the treatment of only an ETA receptor antagonist or RAAS inhibitor alone.

Other suitable modifications and adaptations of the methods of the present disclosure described herein are obvious and may be made using suitable equivalents without departing from the scope of the present disclosure or the embodiments disclosed herein. Having now described the present disclosure in detail, the same will be more clearly understood by reference to the following examples which are included for purposes of illustration only and not intended to limit the scope of the present disclosure. The disclosures of all journal references, U.S. patents and publications referred to herein are hereby incorporated by reference in their entireties.

### EXAMPLES

All experiments were performed using male 30-45 gram CD-1 mice, obtained from Charles River Laboratories, Wilmington, Mass. They were housed under routine vivarium conditions with free food and water access. Surgeries were conducted under deep pentobarbital anesthesia (40-50 mg/Kg IP). Post surgical analgesia was provided with buprenorphine (0.1 mg/Kg IP) at the completion of surgery. All procedures were approved by the institution's IACUC, in accordance with NIH guidelines.

Calculations and Statistics: All values are presented as means±1 SEM. Statistical comparisons were performed by unpaired Student's t test. The mRNA data were generated by competitive RT-PCR, with the results for any given message being expressed as a ratio to the simultaneously determined GAPDH product, used as a housekeeping gene. ChIP data, generated with real time PCR (qPCR) are presented as ng/mg total applied chromatin. The severity of histologic injury, as determined by studying H and E stained kidney sections, was assessed by blinded scoring of slides of 2 week post ischemic kidneys from 5 Atrasentan treated (24 hrs pre-ischemia, and 2 weeks post-ischemia) mice, and from 5 non Atrasentan treated post ischemic controls. The scores were graded on a semiquantitative scale of 1+ to 4+, or least to most severe renal injury observed (based on the extent of proximal tubule necrosis). Continuous variable results were compared by Student's T test. The histologic data were judged by Wilcoxon rank sum test. Significance was judged by a p value of <0.05.

### Example 1

In this example, ET-1 and ETA/ETB receptor expression were quantified following ischemic renal injury, and ET-1 gene chromatin remodeling and RNA polymerase II (Pol II) binding were evaluated. Ten mice were subjected to a midline laparotomy, and the left renal pedicles were exposed and occluded ×30 min using atraumatic microvascular clamps. Uniform ischemia was confirmed by the development of total kidney cyanosis (indicating hypoxia). Body temperature was monitored with an intra-abdominal thermometer and maintained at 37° C. with an external heating source. At the completion of the ischemic period, the vascular clamp was removed, and uniform reperfusion was confirmed by loss of kidney cyanosis. The abdominal incision was then sutured in two layers using 3-0 chromic suture. The mice were then allowed to recover from anesthesia. Ten additional mice, subjected to the same surgical procedure, but not to renal pedicle occlusion, served as sham-operated controls.

At either 24 hrs or 2 weeks post surgery, half of the mice in the post unilateral ischemic group (N=5) or the sham-operated group (N=5) were re-anesthetized and the abdominal incisions were opened. A blood sample was obtained from the inferior vena cava and both kidneys were resected. The kidneys were iced, and the renal cortical samples were obtained with a razor blade, and they were extracted for both total RNA (RNeasy; Qiagen), and total protein. The RNA samples were used to determine the mRNAs for ET-1, and the for ETA and ETB receptors. ET-1 protein concentrations in renal cortical extracts and plasma were determined by ELISA.

To explore whether renal ischemia-reperfusion induces gene-activating histone modifications at the ET-1 gene, potentially increasing ET-1 transcription (such as through ET-1 gene chromatin remodeling and RNA polymerase II (Pol II) binding), renal cortical chromatin extracts were prepared from the following kidneys: kidneys from three sham operated mice (2 weeks post surgery); three 2 week post-ischemic kidneys; and the three corresponding contralateral kidneys. Using a Chromatin immunoprecipitation assay (ChIP), degrees of Pol II binding, histone H3 trimethylation (H3K4m3), histone H3 acetylation (H3K9/K14), and Pol II levels at exon 1 of the ET-1 gene were assessed by real time PCR, as previously described (27-30). In addition, the degree of histone H2A.Z variant exchange at ET-1 exon 1 was also assessed (Naito M, et al., Endotoxin mediates recruitment of RNA polymerase II to target genes in acute renal failure. J Am Soc Nephrol 19: 1321-1330, 2008; Naito M, et al., BRG1 increases transcription of proinflammatory genes in renal ischemia. J Am Soc Nephrol 20: 1787-1796, 2009; Naito M, et al., Renal ischemia-induced cholesterol loading: transcription factor recruitment and chromatin remodeling along the HMG CoA reductase gene. Am J Pathol. 174: 54-62, 2008; Zager R A, et al., Progressive histone alterations and proinflammatory gene activation: consequences of heme protein/iron-mediated proximal tubule injury. Am J Physiol Renal Physiol. 2010 March; 298(3):F827-37. Epub 2009 Dec. 23.) Results were expressed as the amount of Pol II, H3K4m3, H3K9-14 Ac, and H2A.Z at exon 1 per mg of probed chromatin protein. The primer pairs used for qPCR are presented in Table 1.

TABLE 1

| | | |
|---|---|---|
| END 1- | 5′-ACC GCG CTG AGA TCT AAA AA-3′ | 159 bp |
| exon 1 | 5′-CTG CAA AGG GGT CAG AAG AG-3′ | |

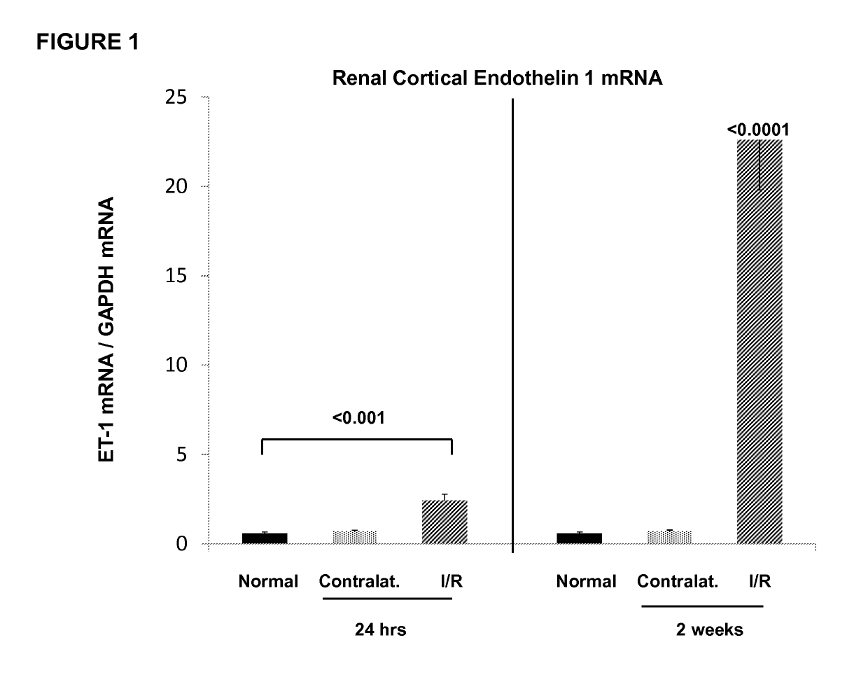

As shown in FIG. 1, within 24 hrs of unilateral ischemic injury a 4 fold increase in ET-1 mRNA was observed, compared to normal kidneys from sham operated controls (p<0.01). By two weeks post ischemia, a marked further ET-1 mRNA increase was observed, reaching values that were 10 fold higher than those observed at the 24 hr time point. These post ischemic ET-1 mRNA increases resulted from ischemia, not surgical stress, given that the contralateral (non-ischemic) kidneys retained normal ET-1 mRNA levels.

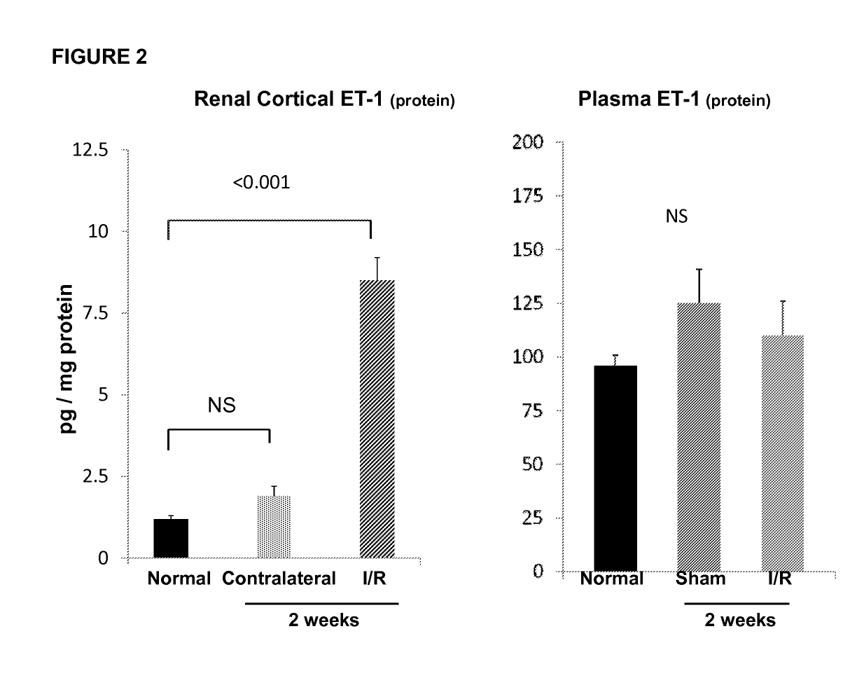

The marked increase in renal cortical ET-1 mRNA was associated with an approximate 8 fold increase in renal cortical ET-1 protein levels (FIG. 2). In contrast, no significant increase in plasma or contralateral kidney ET-1 levels was observed, with values remaining close to those seen in either normal mice or in sham operated surgical controls. This implies that the elevated renal cortical ET-1 protein levels in the two week post ischemic kidneys were a result of increased renal ET-1 production, not uptake from the systemic circulation.

With regard to RNA polymerase II (Pol II) binding and histone modifications at the ET-1 gene following ischemic renal injury, histone modifying enzyme systems can be activated and induce chromatin remodeling at pro-inflammatory genes. These changes include histone H3 trimethylation, acetylation, and histone H2A.Z exchange. (Naito M, et al., Endotoxin mediates recruitment of RNA polymerase II to target genes in acute renal failure. J Am Soc Nephrol 19: 1321-1330, 2008; Naito M, et al., BRG1 increases transcription of proinflammatory genes in renal ischemia. J Am Soc Nephrol 20: 1787-1796, 2009; Naito M, et al., Renal ischemia-induced cholesterol loading: transcription factor recruitment and chromatin remodeling along the HMG CoA reductase gene. Am J Pathol. 174: 54-62, 2008; Zager R A, et al., Progressive histone alterations and proinflammatory gene activation: consequences of heme protein/iron-mediated proximal tubule injury. Am J Physiol Renal Physiol. 2010 March; 298(3): F827-37. Epub 2009 Dec. 23.) By loosening chromatin structure, they facilitate RNA polymerase II (Pol II) recruitment to affected genes and, thus, enhance gene transcription rates.

To assess whether such changes could potentially contribute to the progressive activation of the ET-1 gene post ischemia, ChIP assay was applied to two week post ischemic kidney samples, and significant increases in H3K4m3, H3K9/14 acetylation and H2A.Z levels were observed. The functional significance of these changes was implied by parallel increases in binding of Pol II (the enzyme that drives transcription) to the ET-1 gene. While it cannot be absolutely assumed that there are cause and effect relationships between these histone changes and gene transcription rates, the fact that they are known to be gene activating events in a variety of biologic systems certainly suggest that this is the case.

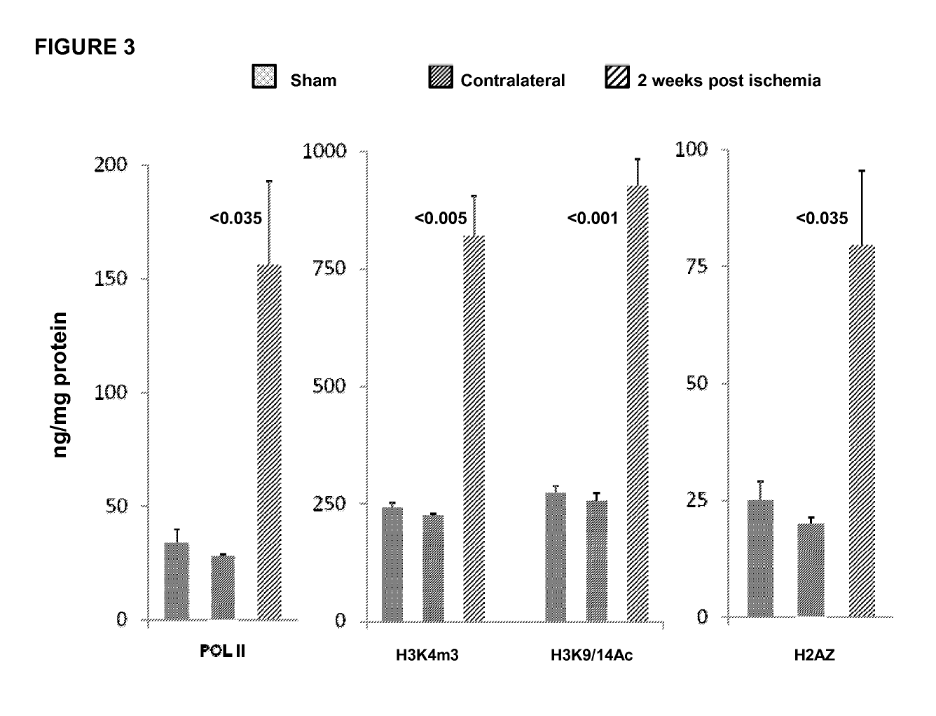

As shown in FIG. 3, the increases in renal cortical ET-1 mRNA at two weeks post ischemia were associated with an approximate 5 fold increase in Pol II binding to the start exon of the ET-1 gene, indicative of a marked increase in gene transcription. Furthermore, increased levels of each of three assessed ‘gene activating’ histone markers (H3K4m3; H3K9Ac, histone variant H2A.Z) corresponded with the increased Pol II levels. Thus, these ChIP data indicate that ischemia-induced acute kidney injury leads to gene-activating histone modifications at the ET-1 gene, which likely contribute to increased gene transcription via increased Pol II recruitment.

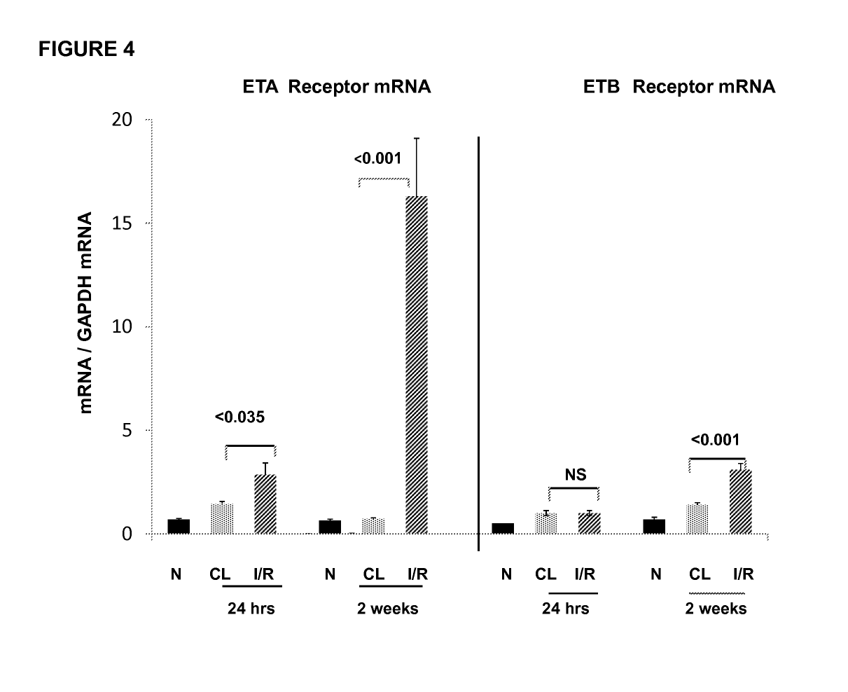

Renal cortical ETA and ETB receptor mRNA expression were also assessed post ischemia. As shown in FIG. 4, a 3 fold increase in ETA receptor mRNA was apparent by 24 hours post ischemia. By 2 weeks post ischemia, a further 8 fold increase in ETA receptor mRNA was observed. Thus, compared to basal values, ETA receptor mRNA levels rose ˜25 fold over the course of the experiment. In sharp contrast, no increase in ETB receptor mRNA was observed at 24 hours post ischemia. By two weeks post ischemia, a significant ETB receptor mRNA increase was observed, but it was quantitatively trivial compare to the ETA mRNA values (25× vs. 2×, respectively).

### Example 2

This example studied whether atrasentan treatment, during pre- and post-ischemic period, confers renal protection. Eighteen mice were subjected to a unilateral ischemia/reperfusion (I/R) protocol as described in Example 1. Nine of the mice received the highly potent and specific ETA receptor antagonist, atrasentan. Atrasentan was administered in the drinking water (25 μg/mL; designed to equate with a dose of ˜5 mg/Kg/day). Atrasentan dosing was started one day before surgery, and continued throughout the remainder of the experiment. Fresh atrasentan was provided ˜2-3× per week for two weeks. The remaining nine mice received only free food and water access, serving as controls.

Upon completion of a two week post ischemic recovery period, the mice were re-anesthetized with pentobarbital, the abdominal incision was re-opened, a terminal blood sample was obtained from the inferior vena cava for BUN and ET-1 analysis, and then the left (post ischemic) kidneys and the right (contralateral) kidneys were removed, and weighed. The degree of post-ischemic loss of renal mass was assessed by comparing the weights of kidneys from sham operated mice, control post-ischemic mice, and post ischemic mice that had received atrasentan treatment. Finally, frontal sections of post-ischemic kidneys were taken from five control mice and five atrasentan-treated mice, fixed in 10% buffered formalin, and used for subsequent histochemical analyses.

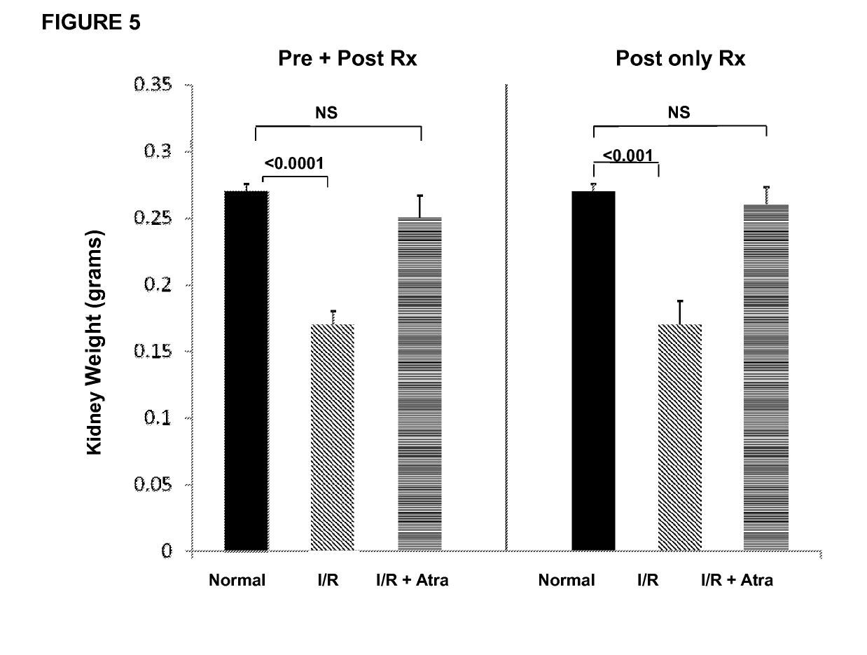

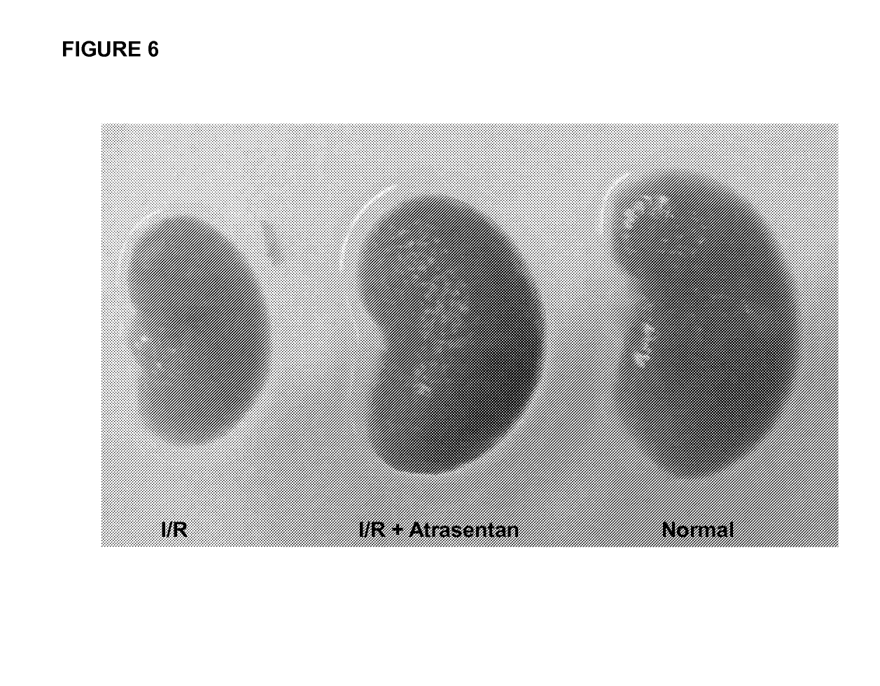

As shown in the left hand panel of FIG. 5, the unilateral I/R injury protocol induced an approximate 45% reduction in renal mass (renal weight), in comparison to the weights of kidneys extracted from sham operated mice (p<0.0001). Sham surgery did not independently affect renal weight, compared to those obtained from normal mice (not shown). Administration of atrasentan, started before renal ischemia and continued throughout the two week post-ischemic period, conferred marked protection, as judged by the fact that the post ischemic kidney weights did not significantly differ from that of normal kidneys. A graphic depiction of this result is presented in FIG. 6: the unilateral ischemic/reperfusion (I/R) kidney (far left) was markedly reduced in size and volume, compared to a normal kidney (far right). In contrast, the pre- and post-ischemia atrasentan treated kidney manifested a near normal size. Thus, the administration of atrasentan before and after renal ischemia can reduce the loss of kidney volume or mass.

### Example 3

This example studies whether atrasentan treatment, restricted to the post-ischemic period, confers renal protection. This experiment was designed to help resolve the issue of when atrasentan was inducing its protective effect. To this end, the same protocol described in Example 2 was repeated, but atrasentan administration was commenced 24 hours after the induction of ischemic damage). At the end of two weeks, the mice were re-anesthetized and the left and right kidneys were weighed. The degree of post ischemic renal weight reduction (the primary endpoint of the above described experiment) was determined. The values were contrasted between the unilateral ischemic kidneys ±atrasentan treatment (N=4, each group), and to values obtained in five kidneys obtained from normal mice.

As shown in the right hand panel of FIG. 5, ischemia without drug treatment caused a 40% reduction in renal weight. Post ischemic atrasentan completely blocked this loss of renal mass, thereby recapitulating the protection seen in the pre+post treatment experiment. This indicates that atrasentan mediated its protective effect during the delayed (>24 hrs) post ischemic period, and not the immediate ischemic/reperfusion injury phase (i.e., during ischemia and 24 hrs of reflow). This suggests that administering atrasentan to a subject suffering ischemic kidney injury at 24 hours or more after that injury is beneficial in providing renal protection from the effects of ischemia and hypoxia.

### Example 4

This example studies the potential effects of atrasentan on renal growth independent of ischemic injury. To ascertain whether atrasentan treatment might impact renal growth or size independent of an effect on renal ischemia/post ischemic injury, renal weights of the contralateral (non ischemic) kidneys from the Example 2 were assessed and compared to ischemic kidneys with and without treatment with atrasentan.

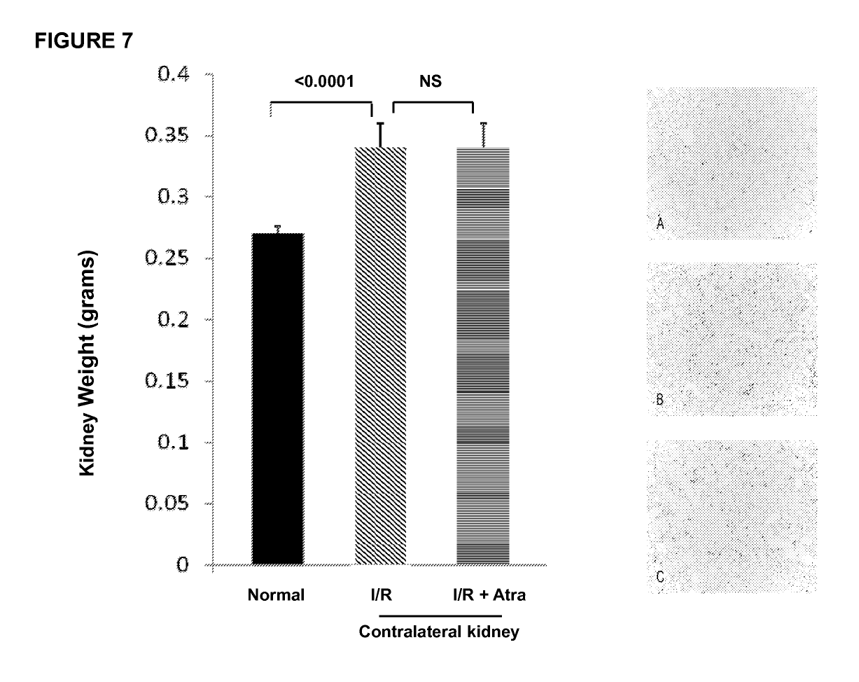

As shown in the left hand panel of FIG. 7, the contralateral kidneys manifested an approximate 25% increase in renal size, compared to normal kidneys. This is consistent with renal hypertrophy in response to a reduction in post-ischemic kidney function, as previously described. (Zager R A, et al., Acute unilateral ischemic renal injury induces progressive renal inflammation, lipid accumulation, histone modification, and “end-stage” kidney disease. Am J Physiol 301: F1334-1345, 2011.) Atrasentan had no effect on this hypertrophic response, given that contralateral kidney weights were essentially identical in the no-drug vs. drug treatment groups.

### Example 5

This example examined renal histology on kidneys from five control mice and five atrasentan-treated mice from Example 2. More particularly, to confirm atrasentan's protective effect against ongoing post ischemic injury, renal histology was examined in kidneys obtained two weeks post ischemia with (combined pre and post) and without atrasentan treatment. Four micron sections were cut and stained with hematoxylin and eosin for overall assessment of the severity of tissue injury. In addition, renal tubular cell proliferation was assessed by immunohistochemical staining for KI-67, a nuclear protein marker of all active cell cycle phases (G1, S, G2, mitosis; but not Go). (Scholzen T, et al., The Ki-67 protein: from the known and the unknown. J Cell Physiol. 182: 311-322, 2000.) The percent of KI-67 cells was determined on whole kidney sections, captured with ScanScope AT (Aperio, Vista, Calif.), and then analyzed with Nuclear Algorithm Spectrum software (version 11.1.1.764; Aperio).

The unilateral ischemia protocol caused marked proximal tubule dropout, extensive interstitial inflammation, ongoing proximal tubule necrosis and extensive intratubular cast formation. These changes were observed throughout the renal cortex and outer medullary stripe. Atrasentan caused a marked reduction in each of these changes. Blinded grading the severity of these changes using a semiquantitative score (1+ to 4+; least to most severe injury observed) revealed a marked diminution in injury scores in the atrasentan group (3.4±0.3 vs 1.7±0.4; p<0.01). Thus, the histologic correlated with the preserved renal mass/renal weight with atrasentan treatment.

As shown in the right hand panel of FIG. 7, KI-67 immunohistochemical staining demonstrated a marked increase in renal tubular cell proliferation in all two week post ischemic kidneys, compared to normal kidneys (p<0.001). The right hand panel depicts KI-67 staining of a normal kidney (A), a two week (left) post ischemic kidney (B), and a two week left post ischemic kidney with pre+post atrasentan treatment (C). The post ischemic kidneys manifested a marked increase in nuclear KI-67 staining, compared to that seen in normal kidneys. Atrasentan did not appear to alter this proliferative response, as denoted by the frequency of KI-67 nuclear staining (% KI-67 nuclear positivity: normal kidneys, 2.4±0.6%; control ischemia, 11.4±0.8%; ischemia+atrasentan, 10.8±1.2%).

### Example 6

This example studies whether atrasentan pre-treatment protects against the acute ischemic injury phase. More particularly, the possibility that atrasentan might mitigate the acute injury phase, and thus, cause a subsequent preservation of renal mass, was explored by subjecting mice to either 22.5 minutes or 25 minutes of bilateral ischemic renal injury in the presence or absence of atrasentan pre-treatment for 24 hours before and for 24 hours after the induction of renal ischemia. The following experiment was undertaken to further assess the time frame in which atrasentan induces protection against ischemic injury. Nine mice were pre-treated for 24 hours with atrasentan and then they were subjected to either 22.5 min (N=6) or 25 min (N=3) of bilateral ischemic injury. An equal number of mice were subjected to the same bilateral ischemia protocols without atrasentan treatment. Atrasentan was continued during the post ischemic period. Twenty four hours later, the mice were re-anesthetized, the abdominal incisions were re-opened, a blood sample was obtained from the inferior vena cava, and the kidneys were resected.

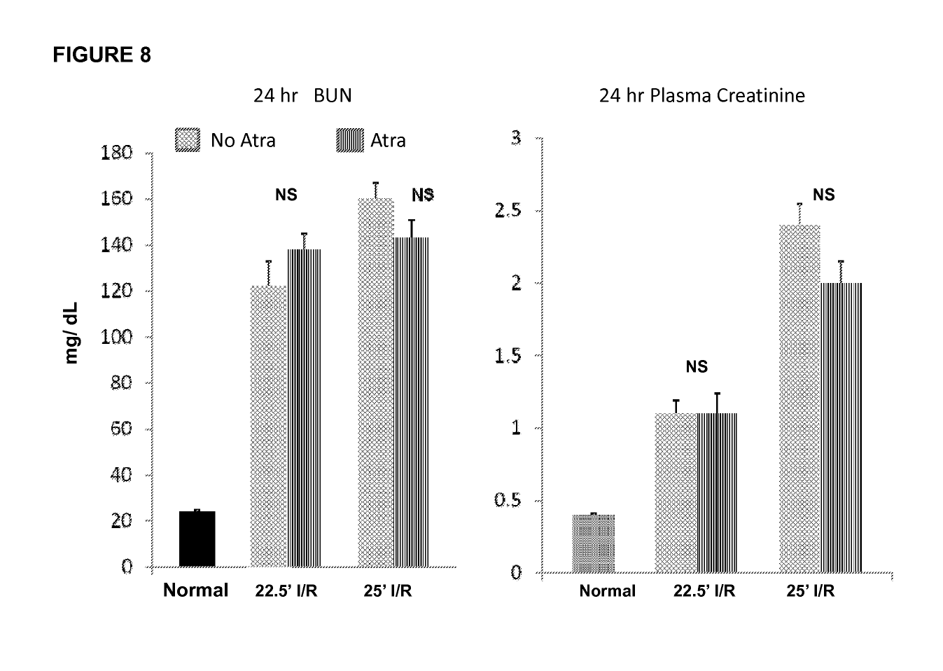

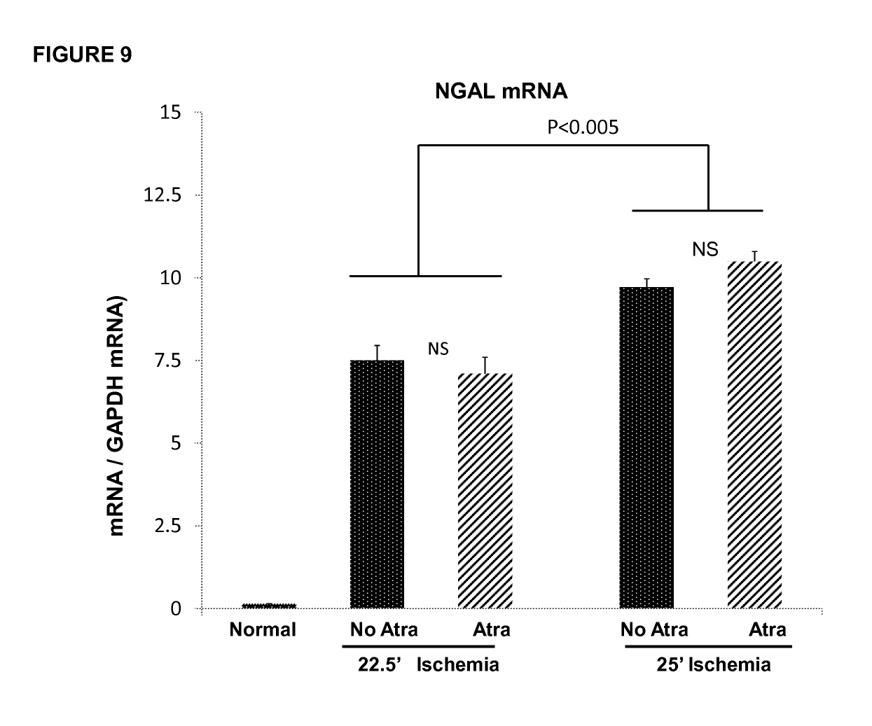

The severity of injury was assessed at 24 hours post ischemia by BUN and plasma creatinine concentrations. Atrasentan failed to mitigate the severity of AKI with either ischemic challenge. As shown in FIG. 8, no protection was observed with either the 22.5 min or the 25 min ischemic challenges (which induced moderate and severe azotemia, respectively). As a second marker of renal injury, renal cortical NGAL mRNA levels were also assessed. Measurement of NGAL mRNA represents a more sensitive biomarker of kidney injury than BUN and Plasma Creatinine measured in FIG. 8 and was measured to investigate the effect of atrasentan on the induction phase of ischemic injury. As shown in FIG. 9, both I/R protocols induced marked NGAL mRNA increases. Significantly higher NGAL mRNA levels being observed with 25 min vs. 22.5 min of ischemia, confirming its ability to serve as a semi-quantitative marker of kidney injury severity. However, in neither instance did atrasentan decrease these NGAL mRNA increases, further supporting the conclusions that: i) atrasentan was unable to block the ischemic injury phase; and ii) that its protective effect, as observed at two weeks post ischemia, was effected during the delayed post ischemic/progressive renal injury phase.

### Example 7

As another assessment of whether atrasentan blocks the early phase of proximal tubule cell injury, cultured proximal tubule (HK-2) cells derived from normal human kidney were incubated in T24 well plates in keratinocyte serum free medium, as previously described in detail. (Iwata M, et al., Sphingosine: a mediator of acute renal tubular injury and subsequent cytoresistance. Proc Natl Acad Sci, USA 9 2: 8970-8974, 1995; Iwata M, et al., Protein synthesis inhibition induces cytoresistance in cultured human proximal tubular (HK-2) cells. Am J Physiol 268: F1154-1163, 1995.) Approximately 18 hrs after seeding, the wells were divided into four groups: (1) control incubation; (2) incubation with atrasentan alone (5 μg/mL; determined by preliminary experiments to be the lowest atrasentan dose that had no independent effect on cell morphology or viability (determined by lactate dehydrogenase, LDH, release); (3) ATP depletion/calcium overload injury, induced with 7.5 μM Antimycin A+10 μM Ca ionophore A23187+20 mM 2-Deoxyglucose, (“CAD”); and 4) CAD+5 μg atrasentan. After eighteen hours of incubation, lethal cell injury was assessed by % LDH release.

It was found that ATP depletion (antimycin/deoxyglucose/Ca ionophore; “CAD”) addition to HK-2 cells induced modest tubular cell death, raising LDH release from a control value of 8.6±0.1% to 22.8±0.2%. Atrasentan did not alter LDH release, either under basal conditions (9.0±0.1%) or with the ATP depletion protocol (23.2±0.3%). This is again consistent with the in vivo data that atrasentan was unable to block an acute ATP depletion injury phase.

### Example 8

This example studies the effect of BQ-788 on post ischemic renal injury. Eight mice were subjected to the unilateral ischemic injury protocol, with half of the mice receiving the ETB specific receptor antagonist BQ-788. The agent was administered at a dose of 3 mmol/Kg subcutaneously each day, beginning 1 day before surgery (a dose chosen to be well in excess of the K1 (100 nM) of ET-1/ETB binding affinity. (Khodorova A, et al., Local injection of a selective endothelin—B receptor antagonist inhibits endothelin-1-induced pain-like behavior and excitation of nociceptors in a naloxone-sensitive manner. J Neurosci 22: 788-7796, 2002; Webber K M, et al., Endothelin induces dopamine release from rat striatum via endothelin-B receptors. Neuroscience. 86: 1173-1180, 1998.) The control unilateral ischemia mice received an equal volume of BQ-788 vehicle (0.1 ml saline injections). Two weeks post-ischemia, the kidneys were harvested and weighed. The degree of post-ischemic loss of renal mass was assessed by comparing the weights of kidneys from sham operated mice, control post-ischemic mice, and post ischemic mice that had received atrasentan treatment.

It was found that BQ-788 failed to decrease the loss of renal injury, as assessed at the two week time point (control ischemia, 0.23 grams; ischemia+BQ-788, 0.24 grams). Thus, these findings point to ET-1's effects on post ischemic renal injury as being mediated through the ETA receptor despite the suggestions that the ETB receptor plays a role in vasodilation induction and possibly has cytoprotective effects. (Kawanabe Y, et al., Endothelin. Cell Molec Life Sci 68:195-203, 2011; Motte S, et al., Endothelin receptor antagonists. Pharmacol Therapeutics 110: 386-414, 2006; Mino N, et al., Protective effect of a selective endothelin receptor antagonist, BQ-123, in ischemic acute renal failure in rats. Eur J Pharmacol 221:77-83, 1992.) If the renoprotective effects exhibit by atrasentan was not due to direct ETA receptor antagonism, but by increasing the availability of ET-1 to the unblocked ETB receptor, then ETB receptor antagonism would be expected to exacerbate renal damage after an ischemic event by blocking the purported cytoprotective effects mediated via an ET-1/ETB receptor interaction and by making more ET-1 available to bind the ETA receptor. However, increased renal damage was not observed when BQ-788 was administered both pre- and post-ischemia, in fact, BQ-788 had no effect on any of the renal parameters measured.

This suggests that selective antagonism of the ETA receptor is desirable over ET-1 inhibition or non-selective ET receptor antagonism as a method for treating acute kidney injury, particularly ischemia-induced kidney injury and hypoxia-induced kidney injury

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

1. A method of treating acute kidney injury in a subject, said method comprising administering to the subject an ETA receptor antagonist.

2. The method of claim 1, wherein the acute kidney injury is an ischemia-induced kidney injury.

3. The method of claim 1, wherein the acute kidney injury is a hypoxia-induced kidney injury.

4. The method of claim 1, wherein the ETA receptor antagonist is atrasentan or a pharmaceutically acceptable salt thereof.

5. The method of claim 1, wherein the ETA receptor antagonist is administered after onset or diagnosis of the acute kidney injury.

6. The method of claim 5, wherein the ETA receptor antagonist is administered at least 24 hours after the acute kidney injury.

7. The method of claim 5, wherein the ETA receptor antagonist is administered after the subject develops clinical acute renal failure.

8. A method of treating ischemia-induced renal injury or hypoxia-induced renal injury in a subject, said method comprising administering to the subject an ETA receptor antagonist.

9. A method of delaying progression to chronic kidney disease in a subject having ischemia-induced renal injury or hypoxia-induced renal injury, said method comprising administering to the subject an ETA receptor antagonist.

10. A method of reversing post-ischemic or post-hypoxic kidney damage in a subject, said method comprising administering to the subject an ETA receptor antagonist.

11. A method of reducing the loss of renal mass or volume in a subject having an ischemia-induced renal injury or hypoxia-induced renal injury, said method comprising administering to the subject an ETA receptor antagonist.

12. A method of reducing an indicator of acute kidney injury in a subject, said method comprising:
diagnosing the subject as having an acute kidney injury;
performing a first measurement of an acute kidney injury indicator;
administering to the subject an ETA receptor antagonist; and
performing a second measurement of the acute kidney injury indicator after the subject has been administered the ETA receptor antagonist for a period of time,
wherein the difference between the first measurement and the second measurement is not significant.

13. The method of claim 12, wherein the acute kidney injury is ischemia-induced or hypoxia-induced.

14. The method of claim 12, wherein the acute kidney injury indicator is kidney mass, kidney volume, glomerular filtration rate, serum creatinine, blood urea nitrogen, or markers of inflammation.

15. A method of diagnosing and treating acute kidney injury in a subject, said method comprising;
measuring a level of an indicator of ischemia-induced renal injury or hypoxia-induced renal injury;
determining whether the measured level indicates ischemia-induced renal injury or hypoxia-induced renal injury; and
administering to the subject suffering from ischemia-induced renal injury or hypoxia-induced renal injury an ETA receptor antagonist.

16. The method of claim 15, wherein the indicator of ischemia-induced renal injury or hypoxia-induced renal injury is urinary tubular injury residue, ET-1 mRNA, ETA receptor mRNA, NGAL mRNA levels, expressed in the kidney, lactate, or markers of inflammation.

17. The method according to claim 8, wherein the ETA receptor antagonist is atrasentan or a pharmaceutically acceptable salt thereof.

18. The method according to claim 8, wherein the ETA receptor antagonist is administered after the kidney injury.

19. The method according to claim 8, wherein the ETA receptor antagonist is administered at least 24 hours after the kidney injury.

20. The method according to claim 8, wherein the ETA receptor antagonist is administered after the subject develops clinical acute renal failure.
